(19) United States
(12) Patent Application Publication
(10) Pub. No.: US 2022/0000764 A1
(43) Pub. Date: Jan. 6, 2022

(54) STABILIZED TERPENE-ENRICHED CANNABINOID EXTRACT AND METHODS OF USE THEREOF

(71) Applicant: Columbia Care LLC, New York, NY (US)

(72) Inventor: Aaron M. Dely, Lighthouse Point, FL (US)

(21) Appl. No.: 17/291,385

(22) PCT Filed: Nov. 6, 2019
(86) PCT No.: PCT/US19/60044

§ 371 Date: May 5, 2021

Related U.S. Application Data
(60) Provisional application No. 62/756,390, filed on Nov. 6, 2018.

(51) Int. Cl. A61K 9/00; A61K 47/14; A61K 31/05; A61K 31/352; A61K 31/015; A61K 9/107

(52) U.S. Cl. CPC A61K 9/008; A61K 47/14; A61K 9/107; A61K 31/352; A61K 31/015; A61K 31/05

(57) ABSTRACT

The invention provides a composition suitable for use in a personal vaporizer, comprising an oil soluble liquid emulsifier base and an extract, wherein the extract comprises a cannabinoid and a terpene, methods of administering such compositions to a subject, and related kits.

### CROSS REFERENCE TO RELATED APPLICATIONS

This application claims the benefit of U.S. Provisional Application No. 62/756,390, filed Nov. 6, 2018, the entire contents of which are incorporated by reference herein.

### FIELD OF THE INVENTION

The invention relates to a composition suitable for use in a personal vaporizer, comprising an oil soluble liquid emulsifier base and an extract, wherein the extract comprises a cannabinoid and a terpene, to methods of administering such compositions to a subject, and to related kits.

### BACKGROUND OF THE INVENTION

The medicinal and psychoactive properties of the cannabis plant have been known for centuries. While it has been illegal in many countries, there is a growing populous to lobby for legalization of its use, especially for medicinal purposes.

Cannabis is believed to provide benefits in the treatment of multiple disorders with safer and fewer serious side effects than most prescription drugs currently used as antiemetics, muscle relaxants, hypnotics and analgesics. A disadvantage in treating patients with cannabis is the psychoactive effect, especially in “naive” cannabis users. Furthermore, there have been reports of unpleasant reactions to cannabis, such as anxiety, panic or hallucinations. It is believed that the undesirable side effects are most commonly associated with higher doses of cannabis and are related to the difficulty in controlling the dosage when the drug is smoked or eaten in cannabis-enriched confectionaries.

Cannabis has also been used to treat the symptoms in patients suffering from serious medical conditions. For example, cannabis has been used to alleviate symptoms associated with cancer, anorexia, AIDS, chronic pain, muscle spasticity, glaucoma, arthritis, migraine and many other illnesses. Cannabis is recognized as having anti-emetic properties and has been successfully used to treat nausea and vomiting in cancer patients undergoing chemotherapy. Cannabis has also been reported in treating the weight loss syndrome of AIDS and for the treatment of glaucoma by reducing intraocular pressure. Cannabis is also known for its muscle relaxing and anti-convulsant effects.

The most prevalent mode of administration of medical cannabis is by smoking. This mode of administration can have adverse effects on the lungs. Cannabis smoke carries more tar and other particulate matter than tobacco and may be a cause of lung diseases including lung cancer. Furthermore, many patients find the act of smoking unappealing, as well as generally unhealthy.

Cannabinoids and other active agents also can be administered in liquid compositions formulated for personal vaporizers—devices that typically utilize a small battery-powered atomizer or heater to turn a liquid into a vapor so that a subject can inhale the vapor. Many personal vaporizers are relatively small and inherently portable and come in a variety of forms. Personal vaporizers can be filled with liquid compositions to be vaporized. Such liquid compositions generally include one or more liquid bases in conjunction with the active drug to be administered. When a solution of liquid base is mixed and heated, the vapor becomes infused with the active ingredient, taking on a variety of therapeutic uses. Typical personal vaporizer liquid bases include propylene glycol, glycerol, or PEG-400 and they are primarily intended to function with the addition of water-soluble additives. Typical known bases such as these are inadequate, however, in that they are unable to emulsify oil-soluble compounds and/or resins for use in a personal vaporizer.

There remains a need in the art, therefore, for an improved non-water soluble personal vaporizer liquid base for the emulsion of oil-soluble compounds.

### SUMMARY OF THE INVENTION

In one aspect, the invention provides a composition suitable for use in a personal vaporizer, comprising: an oil soluble liquid emulsifier base; and an extract, wherein the extract comprises a cannabinoid and a terpene.

In another aspect, the invention provides a method of treating a condition in a subject, comprising administering to the subject a composition suitable for use in a personal vaporizer, the composition comprising an oil soluble liquid emulsifier base and an extract, wherein the extract comprises a cannabinoid and a terpene, e.g., in an amount effective to treat the condition.

In another aspect, the invention provides a kit for administering a composition suitable for use in a personal vaporizer, the kit comprising: a composition comprising an oil soluble liquid emulsifier base and an extract, wherein the extract comprises a cannabinoid and a terpene; a personal vaporizer; and instructions for the use of said kit.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE FIGURES

The present invention will be more fully understood with reference to the following detailed description which is accompanied by drawings.

FIGS. 1-5 contain graphs displaying the cannabinoid profile of compositions suitable for use in a personal vaporizer and containing two cannabinoids in a ratio of 20:1 THC to CBD by weight.

FIGS. 6-11 contain graphs displaying the cannabinoid profile of compositions suitable for use in a personal vaporizer and containing two cannabinoids in a ratio of 1:1 THC to CBD by weight.

FIGS. 12-15 contain graphs displaying the cannabinoid profile of compositions suitable for use in a personal vaporizer and containing two cannabinoids in a ratio of 1:20 THC to CBD by weight.

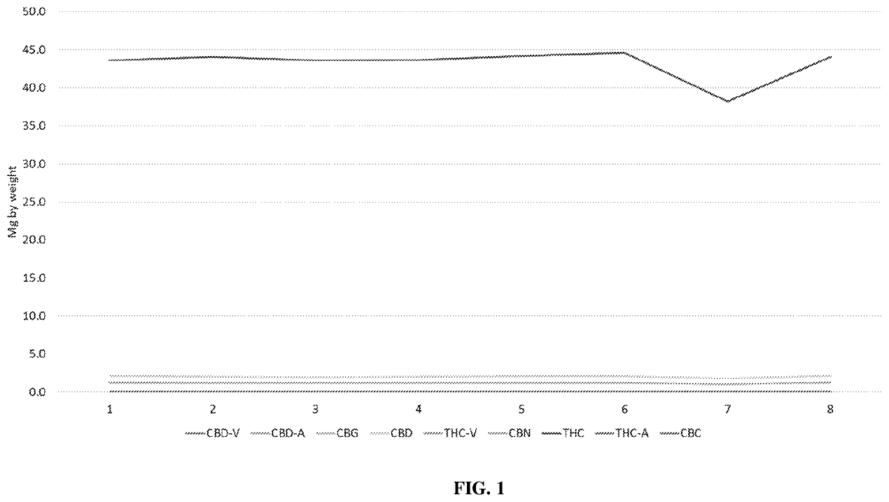

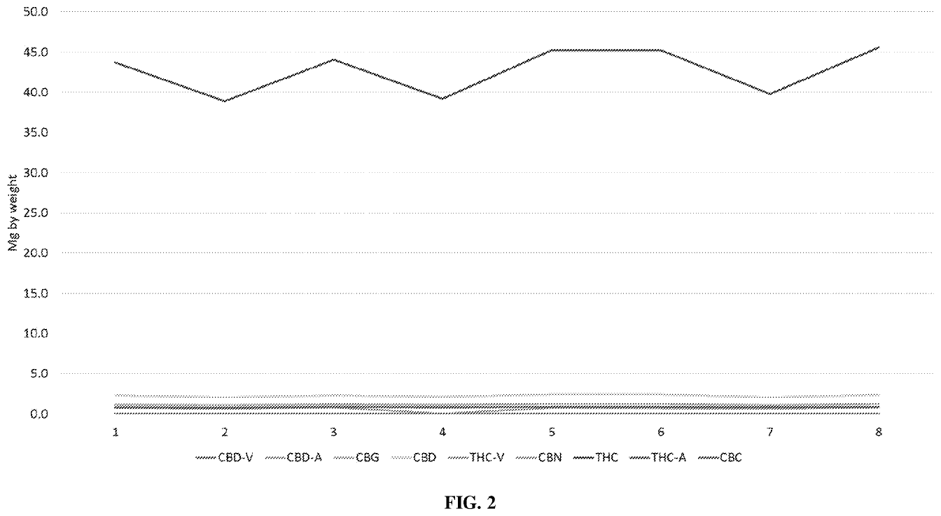

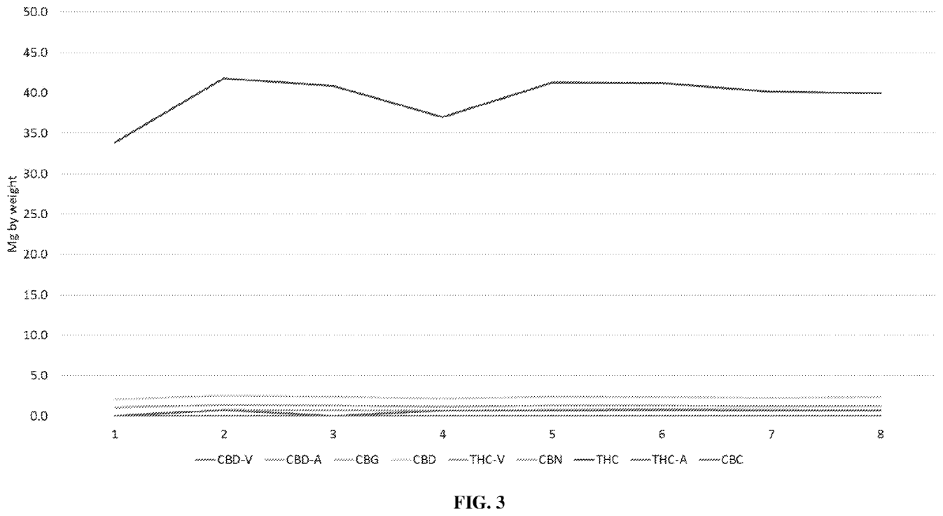

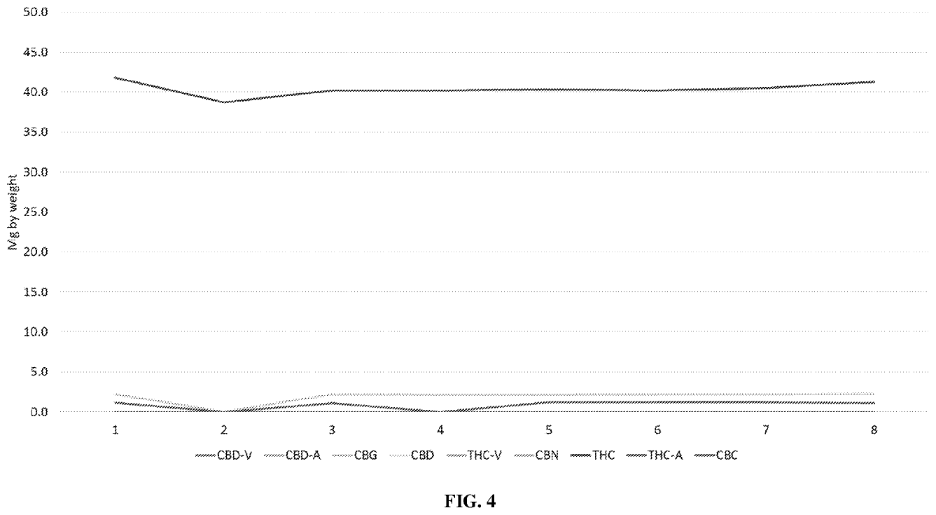

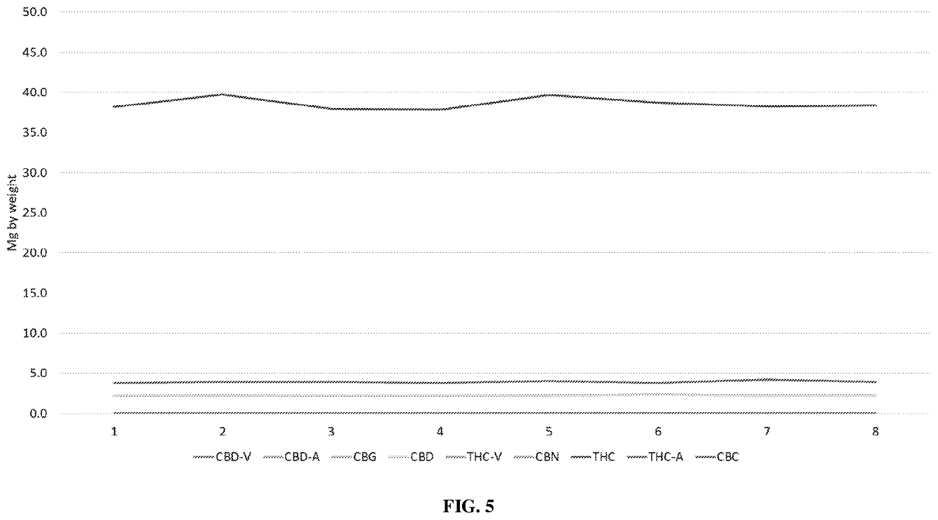

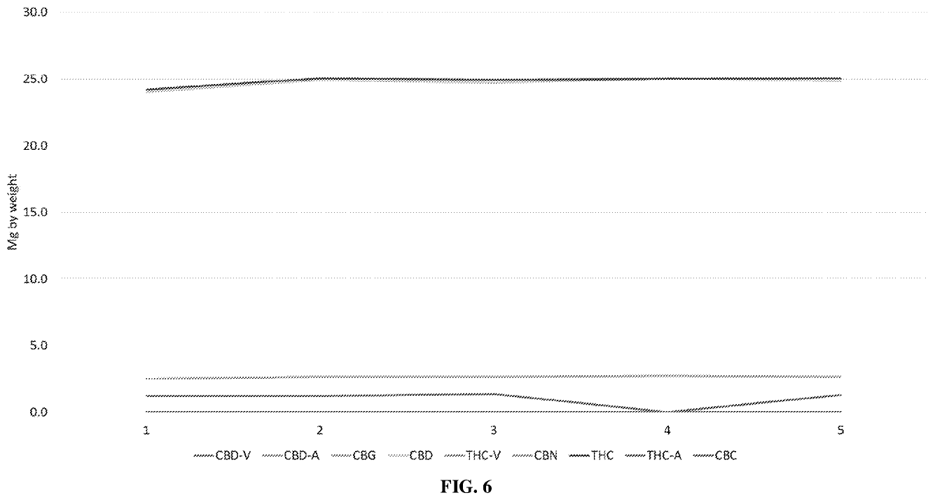

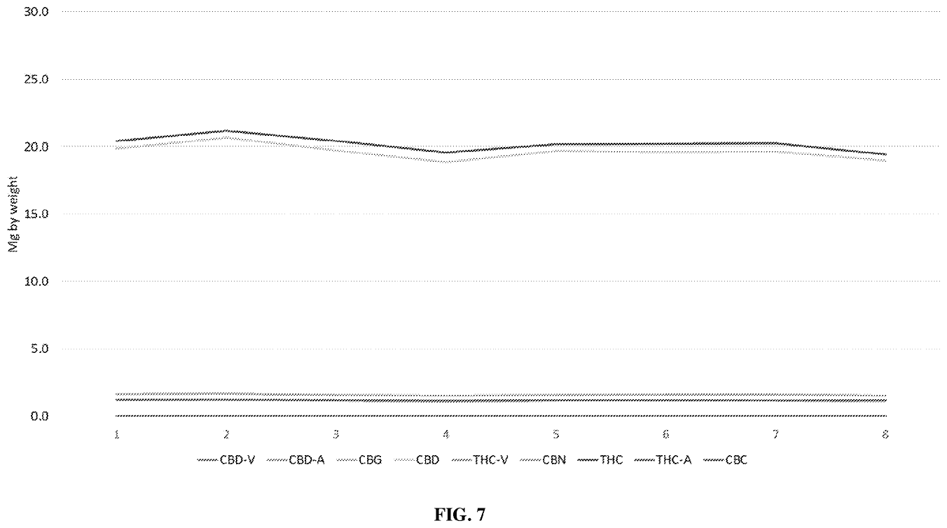

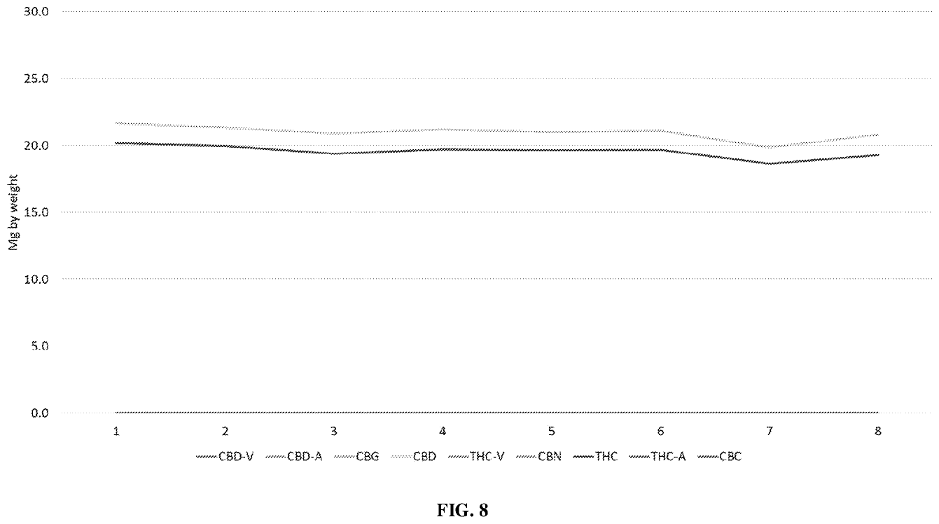

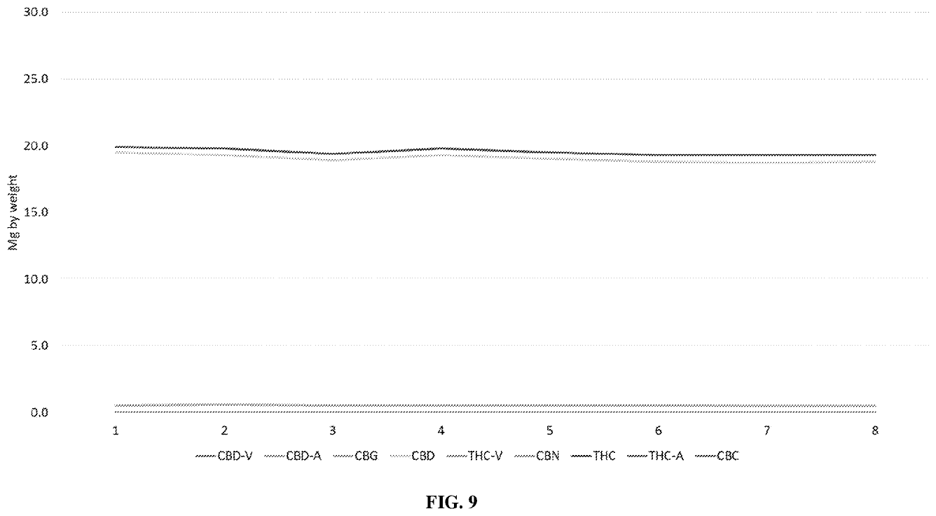

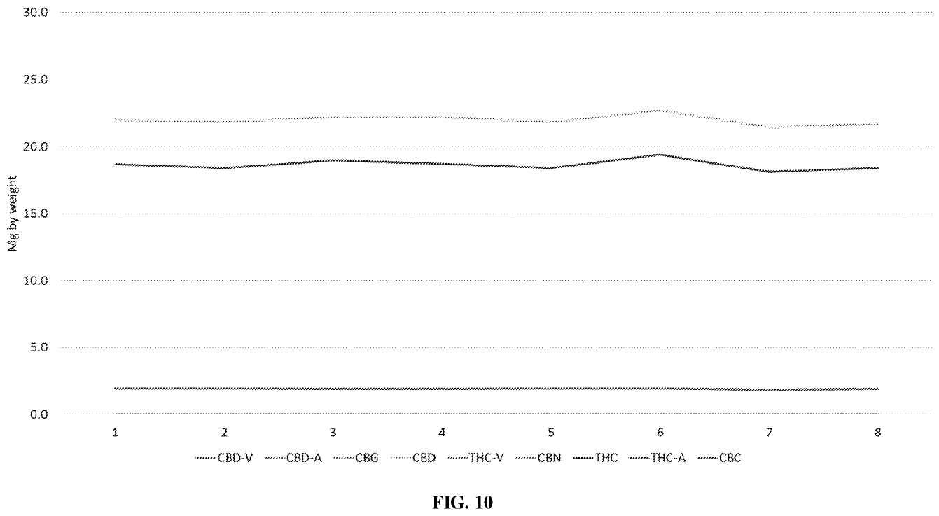

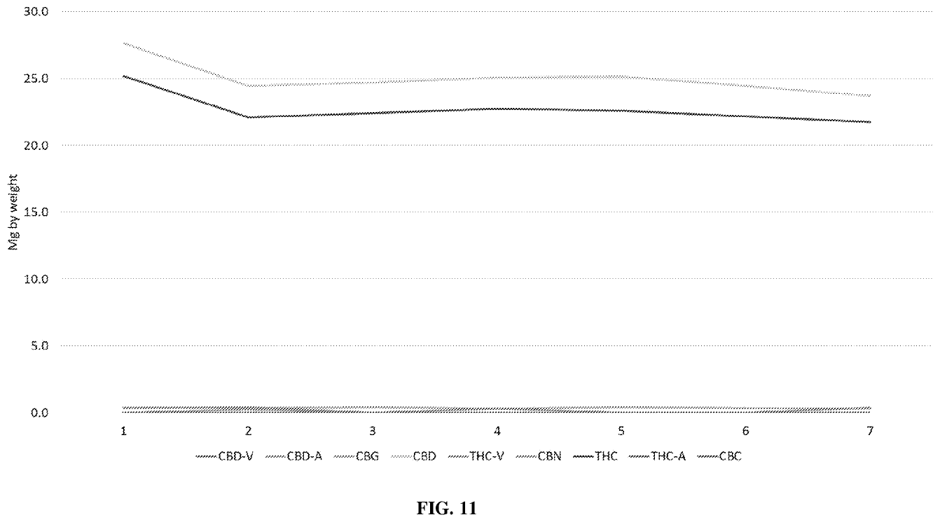

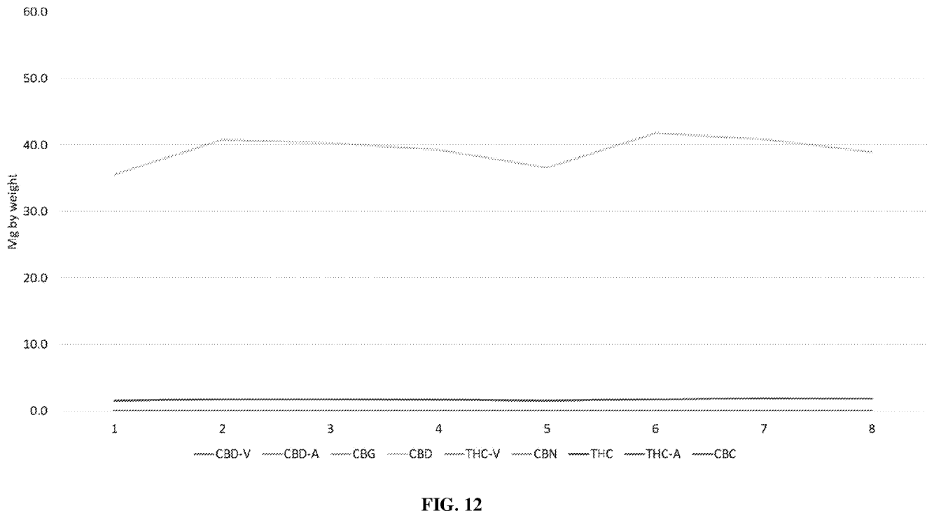

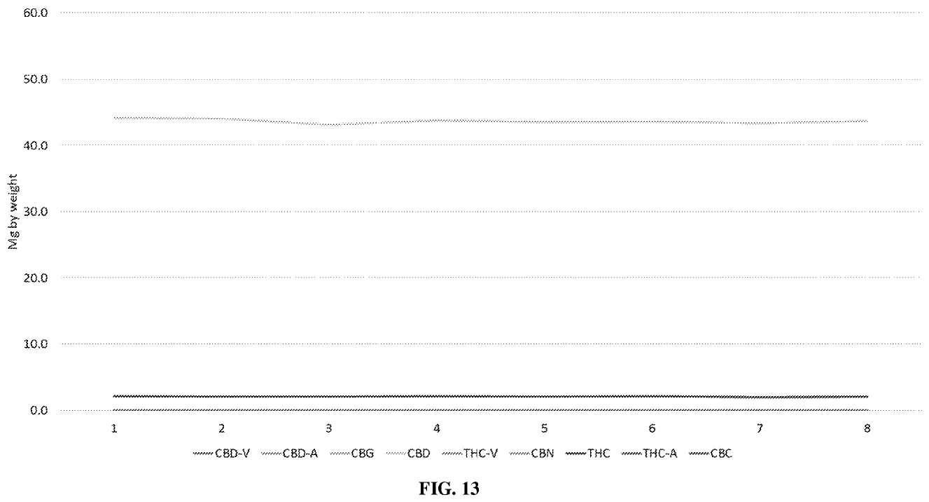

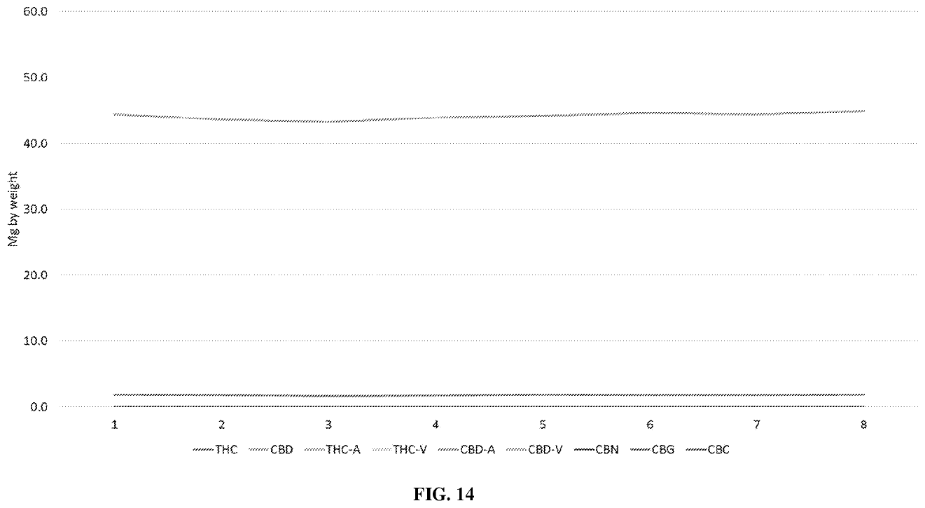

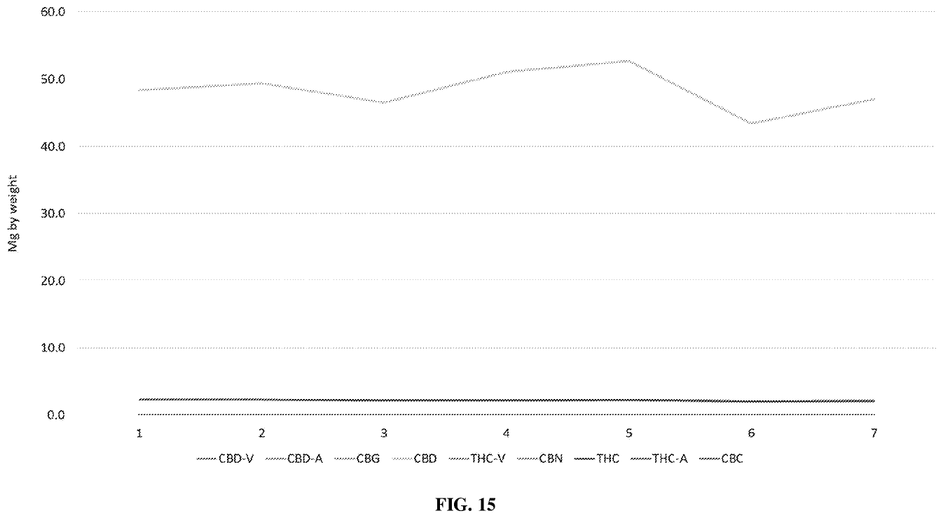

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, other embodiments include from the one particular and/or to the other particular value.

Similarly, when values are expressed as approximations, by use of the antecedent “about,” it is understood that the particular value forms other embodiments. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” “medicament,” or “food product” are used interchangeably herein, as context dictates, to refer to a compound or compounds or composition of matter which, when administered to a subject (human or animal) induces a desired pharmacological and/or physiologic effect by local and/or systemic action.

The terms “subject,” “individual,” and “patient” are used interchangeably herein, and refer to an animal, for example a human, to whom treatment with a composition or formulation or food product in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The formulations described herein can be used to treat any suitable mammal, including primates, such as monkeys and humans, horses, cows, cats, dogs, rabbits, and rodents such as rats and mice. In some embodiments, the mammal to be treated is human. The human can be any human of any age. In certain embodiments, the human is an adult. In certain embodiments, the human can be male, female, middle-aged, adolescent, or elderly. According to any of the methods of the present invention and in certain embodiments, the subject is human.

Conditions and disorders in a subject for which a particular drug, compound, composition, formulation, food product, dietary supplement (or combination thereof) is said herein to be “indicated” are not restricted to conditions and disorders for which that drug or compound or composition or formulation or food product or supplement has been expressly approved by a regulatory authority, but also include other conditions and disorders known or reasonably believed by a physician or other health or nutritional practitioner to be amenable to treatment with that drug or compound or composition or formulation or food product or supplement or combination thereof.

In certain embodiments, provided herein is an improved, non-water soluble personal vaporizer liquid base for the emulsion of oil-soluble compounds. Compositions in accordance with the invention are superior in performance to known compositions for the emulsion of oil-soluble compounds. The invention thus solves or ameliorates, inter alia, the problem of emulsifying oil-soluble compounds and/or resins for use in a personal vaporizer.

In certain embodiments, provided herein is a composition suitable for use in a personal vaporizer, comprising an oil soluble liquid emulsifier base and an extract, wherein the extract comprises a cannabinoid and a terpene.

In certain embodiments, the base comprises a medium chain triglyceride (MCT). In certain embodiments, the MCT comprises a C-6 fatty acid, a C-8 fatty acid, a C-10 fatty acid, a C-12 fatty acid, or a combination thereof.

In certain embodiments, the base comprises glycerol, vegetable glycerine, propylene glycol, ethanol, diglycerol, triglycerol, 1,2-butanediol (BDO), 1,2-pentanediol, 1,2-hexanediol, 1,2-heptanediol, 1,2-octanediol, and mixtures thereof.

Suitable carriers (e.g., a liquid solvent) for the cannabinoids described herein include a medium in which a cannabinoid is soluble at ambient conditions, such that the cannabinoid does not form a solid precipitate. Examples include, but are not limited to vegetable glycerin, glycerol, propylene glycol, trimethylene glycol, water, ethanol and the like, as well as combinations thereof.

In certain embodiments, the cannabinoid comprises tetrahydrocannabinolic acid (THCa), cannabidiolic acid (CBDa), cannabinolic acid (CBNa), cannabichromenic acid (CBCa), tetrahydrocannabinol (THC), cannabinol (CBN), cannabidiol (CBD), cannabichromene (CBC), or a combination thereof. In certain embodiments, the cannabinoid comprises one or more of CBD, THC, THCa, or CBDa.

In some embodiments, the composition has a combination of at least two cannabinoids. In some embodiments, the composition comprises a combination of at least two cannabinoids. In some embodiments, the at least two cannabinoids are selected from Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM) and derivatives thereof, such as THC and CBD. In other embodiments, the at least two cannabinoids are selected from tetrahydrocannabinolic acid (THCa), cannabidiolic acid (CBDa), cannabinolic acid (CBNa), cannabichromenic acid (CBCa), tetrahydrocannabinol (THC), cannabinol (CBN), cannabidiol (CBD), and cannabichromene (CBC), such as THCa and CBDa.

In some embodiments, the at least two cannabinoids are present in a 1:1 proportion by weight. In some embodiments, a first cannabinoid is present in an amount that weighs about between 70 mg and 100 mg and a second cannabinoid is present in an amount that weighs between 70 mg and 100 mg. In some such embodiments, the first cannabinoid is present in an amount that weighs about 87 mg and the second cannabinoid is present in an amount that weighs about 87 mg.

In other embodiments, the at least two cannabinoids are present in a 10:1 proportion by weight. In still other embodiments, the at least two cannabinoids are present in a 20:1 proportion by weight.

In some embodiments, the total weight of cannabinoids present is between 1 and 200 mg. In certain embodiments, a first cannabinoid is present in an amount of about 180 mg and a second cannabinoid is present in an amount that weighs about 9 mg. In other embodiments, a first cannabinoid is present in an amount that weighs about 171 mg and a second cannabinoid is present in an amount that weighs about 7 mg.

As used herein, “terpene” refers to a hydrocarbon or derivative thereof, found as a natural product and biosynthesized by oligomerization of isoprene units. A terpene can be acyclic, monocyclic, bicyclic, or multicyclic. Examples include limonene, pulegone, caryophyllene epoxide, and the like.

In certain embodiments, the terpene comprises either terpenes or terpenoids.

In certain embodiments, the terpene comprises beta-myrcene, myrcene, limonene, beta caryopyllene, caryopyllene oxide, caryophyllene, pinene, pulegone, terpineol, citronellol, linalool, humulene, borneol, bisabolol, camphene, thujone, 1,8-cineole, nerolidol, phytol, geraniol, beta-amyrin, eucalyptol, cycloartenol, isomers thereof or combinations thereof. In certain embodiments, the terpene comprises beta-myrcene, limonene, beta caryopyllene, caryopyllene oxide, terpineol, citronellol, linalool, humulene, beta-amyrin, cycloartenol, or a combination thereof. Any other suitable terpene can also be employed in accordance with the compositions and methods described herein.

According to certain embodiments, said terpenes comprise at least one monoterpene selected from the group consisting of limonene, myrcene, pinene, linalool, geraniol, terpinolene camphene and isomers thereof. According to certain embodiments, said terpenes comprise at least one sesquiterpene selected from the group consisting of nerolidol, caryophyllene, farnesene, zingiberene, vetivazulene, guaiazulene, longifolene, copaene, patchoulol humulene and isomers thereof. According to certain embodiments, said terpenes comprise at least one diterpene selected from the group consisting of phytol, retinal, retinol, phytane, cembrene, sclarene, labdane, abietane, texadiene, stemarene, stemoden and isomers thereof. According to certain embodiments, said terpenes comprise at least one hydroxy-terpene selected from the group consisting of nerolidol, geraniol, linalool, phytol and isomers thereof. As used herein “hydroxy-terpene” refers to a terpene carrying a hydroxyl function.

In certain embodiments, the extract and the base are present in an extract to base ratio of at least 9:1. In other embodiments, the extract and the base are present in an extract to base ratio of about 7:3.

In certain embodiments, the invention provides a method of treating a condition in a subject, comprising administering to the subject a composition suitable for use in a personal vaporizer, the composition comprising an oil soluble liquid emulsifier base and an extract, wherein the extract comprises a cannabinoid and a terpene. In some embodiments, the method comprises administering the composition in an amount effective to treat the condition.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

In certain embodiments, the condition is selected from the group consisting of pain associated with cancer, neuropathic pain, HIV-associated sensory neuropathy, side effects of chemotherapy, symptoms of neurology or a neurodegenerative disease, cancer, hepatitis C, methicillin-resistant Staphylococcus aureus (MRSA), pruritus, psoriasis, asthma, sickle-cell disease, sleep apnea, a digestive disease, collagen-induced arthritis, atherosclerosis and dystonia. In certain embodiments, the side effects of chemotherapy comprise nausea or pain. In certain embodiments, the symptoms of neurology or a neurodegenerative disease comprise Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, or anorexia nervosa. In certain embodiments, the cancer comprises a glioma, a leukemia, a skin tumor, or colorectal cancer. Any other condition, disease, disorder, side effect, or symptom suitable for treatment with a cannabinoid is also within the contemplation of the methods and compositions described herein.

In certain embodiments, the composition is administered in a vaporized form. In certain embodiments, the composition is administered via a personal vaporizer. As discussed above, personal vaporizers are devices that typically utilize a small battery-powered atomizer or heater to turn a liquid into a vapor so that a subject can inhale the vapor. Personal vaporizers suitable for use with the compositions of the present invention include, but are not limited to, electronic cigarettes. Liquid formulations can be loaded into the vaporizer directly (i.e., into a fluid storage compartment thereof) or can be filled into a cartridge (i.e., into a fluid storage compartment thereof) that can then be loaded into the vaporizer. The fluid storage compartment (i.e., of the cartridge or of the vaporizer) is characterized by an internal volume. In certain embodiments, the personal vaporizer is an electronic cigarette.

The heater or heating element may be part of the vaporizer itself or may be part of the cartridge. In some embodiments, the personal vaporizer (e.g., the electronic cigarette) comprises one or more heating elements, a power supply (e.g., a battery), and a fluid storage compartment. In other embodiments, the personal vaporizer (e.g., the electronic cigarette) comprises a power supply adapted for use with a cartridge comprising a fluid storage compartment and one or more heating elements.

In certain embodiments, the cartridge is a container pod. The heater of the vaporizer may be controlled so as to maintain the temperature of the internal volume of the container pod within one or more ranges for a suitable period of time during the stage of herbal extraction. In some embodiments, the temperature within the internal volume of the container pod during herbal extraction may be maintained between 300° F. and 500° F., between 300° F. and 350° F., between 400° F. and 450° F., between 450° F. and 500° F., between 350° F. and 400° F., between 350° F. and 410° F., between 360° F. and 390° F., between 350° F. and 385° F., between 360° F. and 370° F., between 375° F. and 385° F. (e.g., approximately 378° F., approximately 380° F., approximately 382° F., etc.) for at least 5 seconds, at least 10 seconds, at least 15 seconds, at least 20 seconds, at least 30 seconds, at least 60 seconds, or for any other suitable period of time. It can be appreciated that other temperature ranges within the internal volume of the pod during herbal extraction may be maintained for an appropriate period of time.

In certain embodiments, provided herein is a kit for administering a composition suitable for use in a personal vaporizer, the kit comprising: a composition comprising an oil soluble liquid emulsifier base and an extract, wherein the extract comprises a cannabinoid and a terpene; a personal vaporizer; and instructions for the use of said kit. In certain embodiments, the base comprises a medium chain triglyceride (MCT). In certain embodiments, the MCT comprises a C-6 fatty acid, a C-8 fatty acid, a C-10 fatty acid, a C-12 fatty acid, or a combination thereof. In certain embodiments, the cannabinoid comprises tetrahydrocannabinolic acid (THCa), cannabidiolic acid (CBDa), cannabinolic acid (CBNa), cannabichromenic acid (CBCa), tetrahydrocannabinol (THC), cannabinol (CBN), cannabidiol (CBD), cannabichromene (CBC), or a combination thereof, such as one or more of CBD, THC, THCa, or CBDa. In certain embodiments, the terpene comprises beta-myrcene, limonene, beta caryopyllene, caryopyllene oxide, terpineol, citronellol, linalool, humulene, beta-amyrin, cycloartenol, or a combination thereof. In certain embodiments, the extract and the base are present in an extract to base ratio of at least 9:1. In other embodiments, the extract and the base are present in an extract to base ratio of about 7:3.

In certain embodiments, the present invention is directed to a cartridge for use in an electronic cigarette comprising a fluid storage compartment containing the composition as described herein. In certain embodiments, the present invention is directed to a cartridge adapted for use in an electronic cigarette comprising a fluid storage compartment containing the composition as described herein. In certain embodiments, the total volume of the composition present comprises 0.4 ml. In certain embodiments, the cartridge is characterized by a total number of inhalations. In certain embodiments, the total number of inhalations comprises 90 inhalations per cartridge. In certain embodiments, each inhalation comprises 5 seconds. In certain embodiments, the cartridge is configured to deliver one or more inhalations lasting 5 seconds in duration. In certain embodiments, the electronic cigarette comprises a heater, and the heater of the vaporizer can be controlled to maintain the temperature of an internal volume of the cartridge for 5 seconds. In certain embodiments, the heater of the vaporizer is maintained for 5 seconds.

In certain embodiments, the total weight of cannabinoids delivered per 5 second inhalation comprises about 2 mg THC and about 0.1 mg CBD. In certain embodiments, the total weight of cannabinoids delivered per 5 second inhalation comprises 2 mg THC and 0.1 mg CBD. In certain embodiments, the total weight of cannabinoids delivered per 5 second inhalation comprises about 1 mg THC and about 1 mg CBD. In certain embodiments, the total weight of cannabinoids delivered per 5 second inhalation comprises 0.97 mg THC and 0.97 mg CBD. In certain embodiments, the total weight of cannabinoids delivered per 5 second inhalation comprises about .1 mg THC mg THC and about 2 mg CBD. In certain embodiments, the total weight of cannabinoids delivered per 5 second inhalation comprises 0.08 mg THC and 1.9 mg CBD.

Provided herein is a cartridge in an electronic cigarette comprising a fluid storage compartment, wherein the fluid storage compartment stores a cannabinoid liquid formulation comprising a cannabinoid in a biologically acceptable liquid carrier wherein an organic solvent used to form said cannabinoids are characterized by vapor pressure<25 bar at 50° C.

Provided herein is a cartridge in an electronic cigarette comprising a fluid storage compartment, wherein the fluid storage compartment stores a cannabinoid liquid formulation comprising a cannabinoid in a biologically acceptable liquid carrier wherein an organic solvent used to form said cannabinoids are characterized by vapor pressure of about 100 to 10000 bar at 25° C.

Provided herein is a cartridge in an electronic cigarette comprising a fluid storage compartment, wherein the fluid storage compartment stores a cannabinoid liquid formulation comprising a cannabinoid in a biologically acceptable liquid carrier wherein an organic solvent used to form said cannabinoids are further characterized by a melting point<55° C., a boiling point>−165° C., and at least a 15-degree difference between the melting point and the boiling point.

Provided herein is a cartridge in an electronic cigarette comprising a fluid storage compartment, wherein the fluid storage compartment stores a cannabinoid liquid formulation comprising a cannabinoid in a biologically acceptable liquid carrier wherein an organic solvent used to form said cannabinoids are further characterized by a melting point at least 20 degrees lower than an operating temperature of the electronic cigarette, a boiling point no more than 300 degrees lower than the operating temperature of the electronic cigarette, and at least a 15-degree difference between the melting point and the boiling point.

In certain embodiments, the cannabinoid comprises tetrahydrocannabinolic acid (THCa), cannabidiolic acid (CBDa), cannabinolic acid (CBNa), cannabichromenic acid (CBCa), tetrahydrocannabinol (THC), cannabinol (CBN), cannabidiol (CBD), cannabichromene (CBC), or a combination thereof. In certain embodiments, the cannabinoid comprises one or more of CBD, THC, THCa, or CBDa.

In certain embodiments, the extract and the base are present in an extract to base ratio of at least 9:1. In certain embodiments, the extract and the base are present in an extract to base ratio of about 7:3.

In some embodiments described herein, the cannabinoid is a cannabinoid extract that contains one or more of the following: Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM) and derivatives thereof. The cannabinoid may be natural or synthetic.

Methods of preparing cannabinoids extract is well known in the art. The cannabis plants can be grown and harvested, and the cannabinoids extracted through, for example, a CO₂ extraction process.

In some embodiments, the cannabinoids are in equal proportions in the formulation.

The compositions disclosed herein include a composition for daily administration.

Examples of a disease or a disorder that can be treated by the invention include, but not limited to, pain associated with cancer, neuropathic pain and HIV-associated sensory neuropathy, side effects of chemotherapy including nausea and pain, symptoms of neurology and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis and dystonia.

In some embodiments where the disorder is cancer, pain associated with cancer; nausea associated with chemotherapy; or a combination thereof, the composition described herein exerts reduced hallucinatory effects compared to smoking a cannabis containing cigarette or ingesting a cannabis containing foodstuff or other mode of administration with the same amount of active ingredients.

The compositions of the present invention may also contain additional ingredients such as solvents, carriers or excipients.

In some embodiments, cannabinoid of the invention is any member of a group of substances that are structurally related to tetrahydrocannabinol. In some embodiments, the substance can bind to a cannabinoid receptor such as CB1 or CB2 or both (‘THC’). The cannabinoid can be a naturally occurring compound (e.g. present in Cannabis), a compound metabolized by a plant or animal, or a synthetic derivative.

The cannabinoid may be included in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; enol forms.

The cannabinoids of the invention are further meant to encompass natural cannabinoids, natural cannabinoids that have been purified or modified, and synthetically derived cannabinoids, for example, United States Patent Application Publication 2005/0266108, which is hereby incorporated by reference in its entirety, describes a method of purifying cannabinoids obtained from plant material.

The cannabinoids of the invention can be any of 9-tetrahydrocannabinol, 8-tetrahydrocannabinol, (+)-1,1-dimethylheptyl analog of 7-hydroxy-delta-6-tetrahydrocannabinol, 3-(5′-cyano-1′,1′-dimethylpentyl)-1-(4-N-morpholinobutyryloxy) delta 8-tetrahydrocannabinol hydrochloride], dexanabinol, nabilone, levonantradol, or N-(2-hydroxyethyl) hexadecanoamide. In some embodiments, the cannabinoids of the invention can be any of the non-psychotropic cannabinoid 3-dimethylnepty 11 carboxylic acid homologine 8, delta-8-tetrahydrocannabinol.

All patents and literature references cited in the present specification are hereby incorporated by reference in their entireties.

The present invention will be specifically explained by way of examples, but these examples are not intended to limit the present invention.

### EXAMPLE 1

### Stabilized Terpene-Enriched Cannabinoid Extract

Formulations as described above and throughout can comprise at least one cannabinoid, a terpene, and an oil soluble emulsifier, wherein the extract (cannabinoid and terpene) to emulsifier ratio is at least 9:1. In the exemplary formulation provided in Table 1, an extract to emulsifier ratio of about 7:3 is employed.

TABLE 1
Exemplary cannabinoid formulation.

| Ingredient | Percentage by Weight |
|---|---|
| Extract | 70.8 |
| Medium Chain Triglycerides | 29.2 |
| Total | 100 |

It is to be understood that while the present example relates to the composition having certain extract/emulsifier ratios, other ratios (e.g. 9:1, 8:1, 1:1, etc.) are also encompassed by the invention and can be easily prepared using methods known in the art.

Unless defined otherwise, all technical and scientific terms used herein have the same meanings as commonly understood by one of ordinary skill in the art.

Having described preferred embodiments of the present invention with reference to the accompanying drawings, it is to be understood that the present invention is not limited to the above-mentioned embodiments and that various changes and modifications can be affected by one skilled in the art without departing from the spirit or scope of the present invention as defined in the appended claims.

### EXAMPLE 2

### Cannabinoid Profile of Sublingual and Buccal Tinctures

### Materials and Methods

Representative samples of the pharmaceutical formulations in tinctures were diluted/dissolved with organic solvents. A portion of formulation, typically from 10 to 1200 mg, were weighed into a 50-mL centrifuge tube. The amount weighed depended upon the specific product and the declared concentrations of cannabinoids. A Surrogate Standard (SUR) (a pure analyte, which should not be found in any sample, but is similar in nature to the compounds of interest) and 20.0-mL of methanol (MeOH) were added. The solution was mixed well and was either diluted further or used directly for analysis. If necessary, this extract was diluted an additional 2- to 20-fold based on the concentrations of cannabinoids in the sample. The internal standard working diluent (IWD) (a solution of internal standard that is prepared from the internal standard stock diluent and added to all samples at the same concentration) was then added to the extract or dilution thereof, and the potency measurement was made using HPLC-PDA.

The diluted samples fortified with internal standard were injected onto an HPLC. The targeted analytes were separated and subsequently detected online by monitoring UV absorbance using a PDA detector. The separation of ten cannabinoids was achieved on a C18 reverse-phase column 150 mm in length. The limit of quantification for most of the cannabinoids was approximately 0.60 i.t.g/mL. This method was used to quantify the cannabinoid components that are present as low as 0.04% (percent by weight) in the formulations.

Tables 2-6 show the cannabinoid profile for compositions suitable for use in a personal vaporizer and containing two cannabinoids in a ratio of 20:1 THC to CBD by weight.

TABLE 2
Lot Number E170005V
20 THC:1 CBD Vaporization Oil

| | Sample | Sample | Sample | Sample | Sample | Sample | Sample | Sample | |
|---|---|---|---|---|---|---|---|---|---|
| Cannabinoid | 1 | 2 | 3 | 4 | 5 | 6 | 7 | 8 | Average |
| CBD-V | 0.000 | 0.000 | 0.000 | 0.000 | 0.000 | 0.000 | 0.000 | 0.000 | 0.000 |
| CBD-A | 0.000 | 0.000 | 0.000 | 0.000 | 0.000 | 0.000 | 0.000 | 0.000 | 0.000 |
| CBG | 1.280 | 1.170 | 1.180 | 1.170 | 1.190 | 1.220 | 1.030 | 1.260 | 1.188 |
| CBD | 2.140 | 2.050 | 1.980 | 2.040 | 2.080 | 2.070 | 1.770 | 2.130 | 2.033 |
| THC-V | 0.000 | 0.000 | 0.000 | 0.000 | 0.000 | 0.000 | 0.000 | 0.000 | 0.000 |
| CBN | 0.000 | 0.000 | 0.000 | 0.000 | 0.000 | 0.000 | 0.000 | 0.000 | 0.000 |
| THC | 43.570 | 44.040 | 43.570 | 43.650 | 44.190 | 44.580 | 38.190 | 44.030 | 43.228 |
| THC-A | 0.000 | 0.000 | 0.000 | 0.000 | 0.000 | 0.000 | 0.000 | 0.000 | 0.000 |
| CBC | 0.000 | 0.000 | 0.000 | 0.000 | 0.000 | 0.000 | 0.000 | 0.000 | 0.000 |

TABLE 3
Lot Number E170004V
20 THC:1 CBD Vaporization Oil

| | Sample | Sample | Sample | Sample | Sample | Sample | Sample | Sample | |
|---|---|---|---|---|---|---|---|---|---|
| Cannabinoid | 1 | 2 | 3 | 4 | 5 | 6 | 7 | 8 | Average |
| CBD-V | 0.000 | 0.000 | 0.000 | 0.000 | 0.000 | 0.000 | 0.000 | 0.000 | 0.000 |
| CBD-A | 0.000 | 0.000 | 0.000 | 0.000 | 0.000 | 0.000 | 0.000 | 0.000 | 0.000 |
| CBG | 1.160 | 1.080 | 1.180 | 1.140 | 1.250 | 1.240 | 1.050 | 1.250 | 1.169 |
| CBD | 2.310 | 2.100 | 2.340 | 2.140 | 2.440 | 2.420 | 2.100 | 2.370 | 2.278 |
| THC-V | 0.000 | 0.000 | 0.000 | 0.000 | 0.000 | 0.000 | 0.000 | 0.000 | 0.000 |
| CBN | 0.763 | 0.680 | 0.844 | 0.000 | 0.767 | 0.733 | 0.681 | 0.851 | 0.665 |
| THC | 43.720 | 38.920 | 44.040 | 39.220 | 45.230 | 45.260 | 39.770 | 45.570 | 42.716 |
| THC-A | 0.817 | 0.747 | 0.894 | 0.843 | 0.922 | 0.886 | 0.855 | 0.898 | 0.858 |
| CBC | 0.000 | 0.000 | 0.000 | 0.000 | 0.000 | 0.000 | 0.000 | 0.000 | 0.000 |

TABLE 4
Lot Number E160030V
20 THC:1 CBD Vaporization Oil

| | Sample | Sample | Sample | Sample | Sample | Sample | Sample | Sample | |
|---|---|---|---|---|---|---|---|---|---|
| Cannabinoid | 1 | 2 | 3 | 4 | 5 | 6 | 7 | 8 | Average |
| CBD-V | 0.000 | 0.000 | 0.000 | 0.000 | 0.000 | 0.000 | 0.000 | 0.000 | 0.000 |
| CBD-A | 0.000 | 0.000 | 0.000 | 0.000 | 0.000 | 0.000 | 0.000 | 0.000 | 0.000 |
| CBG | 1.080 | 1.370 | 1.290 | 1.130 | 1.320 | 1.310 | 1.220 | 1.280 | 1.250 |
| CBD | 2.000 | 2.580 | 2.380 | 2.130 | 2.350 | 2.340 | 2.280 | 2.320 | 2.298 |
| THC-V | 0.000 | 0.000 | 0.000 | 0.000 | 0.000 | 0.000 | 0.000 | 0.000 | 0.000 |
| CBN | 0.000 | 0.802 | 0.741 | 0.675 | 0.779 | 0.813 | 0.794 | 0.694 | 0.662 |
| THC | 33.840 | 41.820 | 40.850 | 36.980 | 41.250 | 41.240 | 40.170 | 39.940 | 39.511 |
| THC-A | 0.000 | 0.000 | 0.000 | 0.000 | 0.000 | 0.000 | 0.000 | 0.000 | 0.000 |
| CBC | 0.000 | 0.726 | 0.000 | 0.684 | 0.660 | 0.694 | 0.663 | 0.679 | 0.513 |

TABLE 5
Lot Number E160017A
20 THC:1 CBD Vaporization Oil

| | Sample | Sample | Sample | Sample | Sample | Sample | Sample | Sample | |
|---|---|---|---|---|---|---|---|---|---|
| Cannabinoid | 1 | 2 | 3 | 4 | 5 | 6 | 7 | 8 | Average |
| CBD-V | 0.000 | 0.000 | 0.000 | 0.000 | 0.000 | 0.000 | 0.000 | 0.000 | 0.000 |
| CBD-A | 0.000 | 0.000 | 0.000 | 0.000 | 0.000 | 0.000 | 0.000 | 0.000 | 0.000 |
| CBG | 0.000 | 0.000 | 0.000 | 0.000 | 0.000 | 0.000 | 0.000 | 0.000 | 0.000 |
| CBD | 2.180 | 0.000 | 2.190 | 2.140 | 2.140 | 2.210 | 2.170 | 2.260 | 1.911 |
| THC-V | 0.000 | 0.000 | 0.000 | 0.000 | 0.000 | 0.000 | 0.000 | 0.000 | 0.000 |
| CBN | 0.000 | 0.000 | 0.000 | 0.000 | 0.000 | 0.000 | 0.000 | 0.000 | 0.000 |
| THC | 41.800 | 38.700 | 40.200 | 40.200 | 40.300 | 40.200 | 40.500 | 41.300 | 40.400 |
| THC-A | 1.110 | 0.000 | 1.100 | 0.000 | 1.180 | 1.170 | 1.170 | 1.100 | 0.854 |
| CBC | 0.000 | 0.000 | 0.000 | 0.000 | 0.000 | 0.000 | 0.000 | 0.000 | 0.000 |

TABLE 6
Lot Number E160011A
20 THC:1 CBD Vaporization Oil

| | Sample | Sample | Sample | Sample | Sample | Sample | Sample | Sample | |
|---|---|---|---|---|---|---|---|---|---|
| Cannabinoid | 1 | 2 | 3 | 4 | 5 | 6 | 7 | 8 | Average |
| CBD-V | 0.000 | 0.000 | 0.000 | 0.000 | 0.000 | 0.000 | 0.000 | 0.000 | 0.000 |
| CBD-A | 0.000 | 0.000 | 0.000 | 0.000 | 0.000 | 0.000 | 0.000 | 0.000 | 0.000 |
| CBG | 0.000 | 0.000 | 0.000 | 0.000 | 0.000 | 0.000 | 0.000 | 0.000 | 0.000 |
| CBD | 2.180 | 2.250 | 2.220 | 2.190 | 2.270 | 2.360 | 2.230 | 2.240 | 2.243 |
| THC-V | 0.000 | 0.000 | 0.000 | 0.000 | 0.000 | 0.000 | 0.000 | 0.000 | 0.000 |
| CBN | 0.000 | 0.000 | 0.000 | 0.000 | 0.000 | 0.000 | 0.000 | 0.000 | 0.000 |
| THC | 38.170 | 39.740 | 37.960 | 37.820 | 39.690 | 38.700 | 38.220 | 38.370 | 38.584 |
| THC-A | 3.790 | 3.890 | 3.890 | 3.820 | 4.030 | 3.820 | 4.220 | 3.940 | 3.925 |
| CBC | 0.000 | 0.000 | 0.000 | 0.000 | 0.000 | 0.000 | 0.000 | 0.000 | 0.000 |

Tables 7-12 show the cannabinoid profile for compositions suitable for use in a personal vaporizer and containing two cannabinoids in a ratio of 1:1 THC to CBD by weight.

TABLE 7

| | Sample | Sample | Sample | Sample | Sample | |
|---|---|---|---|---|---|---|
| Cannabinoid | 1 | 2 | 3 | 4 | 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 2.500 | 2.650 | 2.620 | 2.710 | 2.620 | 2.620 |
| CBD | 24.000 | 24.890 | 24.680 | 25.030 | 24.830 | 24.686 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 24.210 | 25.050 | 24.950 | 25.010 | 25.030 | 24.850 |
| THC-A | 0.000 | 0.000 | 0.000 | 0.000 | 0.000 | 0.000 |
| CBC | 1.210 | 1.230 | 1.350 | 0.000 | 1.290 | 1.016 |

TABLE 8
Lot Number E160037V
1 THC:1 CBD Vaporization Oil

| | Sample | Sample | Sample | Sample | Sample | Sample | Sample | Sample | |
|---|---|---|---|---|---|---|---|---|---|
| Cannabinoid | 1 | 2 | 3 | 4 | 5 | 6 | 7 | 8 | Average |
| CBD-V | 0.000 | 0.000 | 0.000 | 0.000 | 0.000 | 0.000 | 0.000 | 0.000 | 0.000 |
| CBD-A | 1.160 | 1.210 | 1.150 | 1.070 | 1.140 | 1.160 | 1.130 | 1.120 | 1.143 |
| CBG | 1.640 | 1.670 | 1.570 | 1.530 | 1.570 | 1.600 | 1.590 | 1.520 | 1.586 |
| CBD | 19.840 | 20.660 | 19.710 | 18.860 | 19.690 | 19.590 | 19.650 | 18.950 | 19.619 |
| THC-V | 0.000 | 0.000 | 0.000 | 0.000 | 0.000 | 0.000 | 0.000 | 0.000 | 0.000 |
| CBN | 0.000 | 0.000 | 0.000 | 0.000 | 0.000 | 0.000 | 0.000 | 0.000 | 0.000 |
| THC | 20.420 | 21.180 | 20.410 | 19.570 | 20.170 | 20.220 | 20.250 | 19.420 | 20.205 |
| THC-A | 0.000 | 0.000 | 0.000 | 0.000 | 0.000 | 0.000 | 0.000 | 0.000 | 0.000 |
| CBC | 1.200 | 1.210 | 1.160 | 1.140 | 1.190 | 1.140 | 1.150 | 1.160 | 1.169 |

TABLE 9
Lot Number E160031V
1 THC:1 CBD Vaporization Oil

| | Sample | Sample | Sample | Sample | Sample | Sample | Sample | Sample | |
|---|---|---|---|---|---|---|---|---|---|
| Cannabinoid | 1 | 2 | 3 | 4 | 5 | 6 | 7 | 8 | Average |
| CBD-V | 0.000 | 0.000 | 0.000 | 0.000 | 0.000 | 0.000 | 0.000 | 0.000 | 0.000 |
| CBD-A | 0.000 | 0.000 | 0.000 | 0.000 | 0.000 | 0.000 | 0.000 | 0.000 | 0.000 |
| CBG | 0.000 | 0.000 | 0.000 | 0.000 | 0.000 | 0.000 | 0.000 | 0.000 | 0.000 |
| CBD | 21.660 | 21.350 | 20.900 | 21.220 | 20.990 | 21.090 | 19.870 | 20.820 | 20.988 |
| THC-V | 0.000 | 0.000 | 0.000 | 0.000 | 0.000 | 0.000 | 0.000 | 0.000 | 0.000 |
| CBN | 0.000 | 0.000 | 0.000 | 0.000 | 0.000 | 0.000 | 0.000 | 0.000 | 0.000 |
| THC | 20.180 | 19.960 | 19.400 | 19.720 | 19.650 | 19.670 | 18.650 | 19.300 | 19.566 |
| THC-A | 0.000 | 0.000 | 0.000 | 0.000 | 0.000 | 0.000 | 0.000 | 0.000 | 0.000 |
| CBC | 0.000 | 0.000 | 0.000 | 0.000 | 0.000 | 0.000 | 0.000 | 0.000 | 0.000 |

TABLE 10
Lot Number E160018A
1 THC:1 CBD Vaporization Oil

| | Sample | Sample | Sample | Sample | Sample | Sample | Sample | Sample | |
|---|---|---|---|---|---|---|---|---|---|
| Cannabinoid | 1 | 2 | 3 | 4 | 5 | 6 | 7 | 8 | Average |
| CBD-V | 0.000 | 0.000 | 0.000 | 0.000 | 0.000 | 0.000 | 0.000 | 0.000 | 0.000 |
| CBD-A | 0.000 | 0.000 | 0.000 | 0.000 | 0.000 | 0.000 | 0.000 | 0.000 | 0.000 |
| CBG | 0.520 | 0.560 | 0.520 | 0.540 | 0.540 | 0.540 | 0.510 | 0.510 | 0.530 |
| CBD | 19.500 | 19.300 | 18.900 | 19.300 | 19.000 | 18.800 | 18.700 | 18.800 | 19.038 |
| THC-V | 0.000 | 0.000 | 0.000 | 0.000 | 0.000 | 0.000 | 0.000 | 0.000 | 0.000 |
| CBN | 0.000 | 0.000 | 0.000 | 0.000 | 0.000 | 0.000 | 0.000 | 0.000 | 0.000 |
| THC | 19.900 | 19.800 | 19.400 | 19.800 | 19.500 | 19.300 | 19.300 | 19.300 | 19.538 |
| THC-A | 0.000 | 0.000 | 0.000 | 0.000 | 0.000 | 0.000 | 0.000 | 0.000 | 0.000 |
| CBC | 0.000 | 0.000 | 0.000 | 0.000 | 0.000 | 0.000 | 0.000 | 0.000 | 0.000 |

TABLE 11
Lot Number E160012A
1 THC:1 CBD Vaporization Oil

| | Sample | Sample | Sample | Sample | Sample | Sample | Sample | Sample | |
|---|---|---|---|---|---|---|---|---|---|
| Cannabinoid | 1 | 2 | 3 | 4 | 5 | 6 | 7 | 8 | Average |
| CBD-V | 0.000 | 0.000 | 0.000 | 0.000 | 0.000 | 0.000 | 0.000 | 0.000 | 0.000 |
| CBD-A | 0.000 | 0.000 | 0.000 | 0.000 | 0.000 | 0.000 | 0.000 | 0.000 | 0.000 |
| CBG | 0.000 | 0.000 | 0.000 | 0.000 | 0.000 | 0.000 | 0.000 | 0.000 | 0.000 |
| CBD | 22.000 | 21.820 | 22.200 | 22.210 | 21.810 | 22.690 | 21.430 | 21.690 | 21.981 |
| THC-V | 0.000 | 0.000 | 0.000 | 0.000 | 0.000 | 0.000 | 0.000 | 0.000 | 0.000 |
| CBN | 0.000 | 0.000 | 0.000 | 0.000 | 0.000 | 0.000 | 0.000 | 0.000 | 0.000 |
| THC | 18.690 | 18.410 | 18.970 | 18.700 | 18.380 | 19.380 | 18.120 | 18.410 | 18.633 |
| THC-A | 1.910 | 1.920 | 1.890 | 1.880 | 1.920 | 1.940 | 1.800 | 1.890 | 1.894 |
| CBC | 0.000 | 0.000 | 0.000 | 0.000 | 0.000 | 0.000 | 0.000 | 0.000 | 0.000 |

TABLE 12
Lot Number E160008A
1 THC:1 CBD Vaporization Oil

| | Sample | Sample | Sample | Sample | Sample | Sample | Sample | |
|---|---|---|---|---|---|---|---|---|
| Cannabinoid | 1 | 2 | 3 | 4 | 5 | 6 | 7 | Average |
| CBD-V | 0.000 | 0.000 | 0.000 | 0.000 | 0.000 | 0.000 | 0.000 | 0.000 |
| CBD-A | 0.000 | 0.000 | 0.000 | 0.000 | 0.000 | 0.000 | 0.000 | 0.000 |
| CBG | 0.400 | 0.370 | 0.430 | 0.340 | 0.440 | 0.370 | 0.340 | 0.384 |
| CBD | 27.630 | 24.450 | 24.700 | 25.080 | 25.140 | 24.440 | 23.700 | 25.020 |
| THC-V | 0.350 | 0.420 | 0.000 | 0.340 | 0.000 | 0.000 | 0.410 | 0.217 |
| CBN | 0.000 | 0.270 | 0.000 | 0.000 | 0.000 | 0.000 | 0.000 | 0.039 |
| THC | 25.200 | 22.080 | 22.410 | 22.750 | 22.580 | 22.180 | 21.750 | 22.707 |
| THC-A | 0.000 | 0.000 | 0.000 | 0.000 | 0.000 | 0.000 | 0.000 | 0.000 |
| CBC | 0.000 | 0.000 | 0.000 | 0.000 | 0.000 | 0.000 | 0.000 | 0.000 |

Tables 13-16 show the cannabinoid profile for compositions suitable for use in a personal vaporizer and containing two cannabinoids in a ratio of 1:20 THC to CBD by weight.

TABLE 13
Lot Number E160032V
1 THC:20 CBD Vaporization Oil

| | Sample | Sample | Sample | Sample | Sample | Sample | Sample | Sample | |
|---|---|---|---|---|---|---|---|---|---|
| Cannabinoid | 1 | 2 | 3 | 4 | 5 | 6 | 7 | 8 | Average |
| CBD-V | 0.000 | 0.000 | 0.000 | 0.000 | 0.000 | 0.000 | 0.000 | 0.000 | 0.000 |
| CBD-A | 0.000 | 0.000 | 0.000 | 0.000 | 0.000 | 0.000 | 0.000 | 0.000 | 0.000 |
| CBG | 0.000 | 0.000 | 0.000 | 0.000 | 0.000 | 0.000 | 0.000 | 0.000 | 0.000 |
| CBD | 35.520 | 40.760 | 40.290 | 39.310 | 36.590 | 41.760 | 40.860 | 38.960 | 39.256 |
| THC-V | 0.000 | 0.000 | 0.000 | 0.000 | 0.000 | 0.000 | 0.000 | 0.000 | 0.000 |
| CBN | 0.000 | 0.000 | 0.000 | 0.000 | 0.000 | 0.000 | 0.000 | 0.000 | 0.000 |
| THC | 1.530 | 1.720 | 1.700 | 1.640 | 1.510 | 1.740 | 1.860 | 1.770 | 1.684 |
| THC-A | 0.000 | 0.000 | 0.000 | 0.000 | 0.000 | 0.000 | 0.000 | 0.000 | 0.000 |
| CBC | 0.000 | 0.000 | 0.000 | 0.000 | 0.000 | 0.000 | 0.000 | 0.000 | 0.000 |

TABLE 14
Lot Number E160019A
1 THC:20 CBD Vaporization Oil

| | Sample | Sample | Sample | Sample | Sample | Sample | Sample | Sample | |
|---|---|---|---|---|---|---|---|---|---|
| Cannabinoid | 1 | 2 | 3 | 4 | 5 | 6 | 7 | 8 | Average |
| CBD-V | 0.000 | 0.000 | 0.000 | 0.000 | 0.000 | 0.000 | 0.000 | 0.000 | 0.000 |
| CBD-A | 0.000 | 0.000 | 0.000 | 0.000 | 0.000 | 0.000 | 0.000 | 0.000 | 0.000 |
| CBG | 0.000 | 0.000 | 0.000 | 0.000 | 0.000 | 0.000 | 0.000 | 0.000 | 0.000 |
| CBD | 44.190 | 44.070 | 43.110 | 43.790 | 43.580 | 43.590 | 43.320 | 43.690 | 43.668 |
| THC-V | 0.000 | 0.000 | 0.000 | 0.000 | 0.000 | 0.000 | 0.000 | 0.000 | 0.000 |
| CBN | 0.000 | 0.000 | 0.000 | 0.000 | 0.000 | 0.000 | 0.000 | 0.000 | 0.000 |
| THC | 2.130 | 2.080 | 2.050 | 2.150 | 2.050 | 2.130 | 2.020 | 2.050 | 2.083 |
| THC-A | 0.000 | 0.000 | 0.000 | 0.000 | 0.000 | 0.000 | 0.000 | 0.000 | 0.000 |
| CBC | 0.000 | 0.000 | 0.000 | 0.000 | 0.000 | 0.000 | 0.000 | 0.000 | 0.000 |

TABLE 15
Lot Number E160013A
1 THC:20 CBD Vaporization Oil

| | Sample | Sample | Sample | Sample | Sample | Sample | Sample | Sample | |
|---|---|---|---|---|---|---|---|---|---|
| Cannabinoid | 1 | 2 | 3 | 4 | 5 | 6 | 7 | 8 | Average |
| THC | 1.830 | 1.760 | 1.650 | 1.700 | 1.850 | 1.760 | 1.770 | 1.880 | 1.775 |
| CBD | 44.380 | 43.640 | 43.250 | 43.930 | 44.200 | 44.600 | 44.400 | 44.930 | 44.166 |
| THC-A | 0.000 | 0.000 | 0.000 | 0.000 | 0.000 | 0.000 | 0.000 | 0.000 | 0.000 |
| THC-V | 0.000 | 0.000 | 0.000 | 0.000 | 0.000 | 0.000 | 0.000 | 0.000 | 0.000 |
| CBD-A | 0.000 | 0.000 | 0.000 | 0.000 | 0.000 | 0.000 | 0.000 | 0.000 | 0.000 |
| CBD-V | 0.000 | 0.000 | 0.000 | 0.000 | 0.000 | 0.000 | 0.000 | 0.000 | 0.000 |
| CBN | 0.000 | 0.000 | 0.000 | 0.000 | 0.000 | 0.000 | 0.000 | 0.000 | 0.000 |
| CBG | 0.000 | 0.000 | 0.000 | 0.000 | 0.000 | 0.000 | 0.000 | 0.000 | 0.000 |
| CBC | 0.000 | 0.000 | 0.000 | 0.000 | 0.000 | 0.000 | 0.000 | 0.000 | 0.000 |

TABLE 16
Lot Number E160008A
1 THC:1 CBD Vaporization Oil

| | Sample | Sample | Sample | Sample | Sample | Sample | Sample | |
|---|---|---|---|---|---|---|---|---|
| Cannabinoid | 1 | 2 | 3 | 4 | 5 | 6 | 7 | Average |
| CBD-V | 0.000 | 0.000 | 0.000 | 0.000 | 0.000 | 0.000 | 0.000 | 0.000 |
| CBD-A | 0.000 | 0.000 | 0.000 | 0.000 | 0.000 | 0.000 | 0.000 | 0.000 |
| CBG | 0.000 | 0.000 | 0.000 | 0.000 | 0.000 | 0.000 | 0.000 | 0.000 |
| CBD | 48.380 | 49.410 | 46.510 | 51.060 | 52.730 | 43.420 | 47.030 | 48.363 |
| THC-V | 0.000 | 0.000 | 0.000 | 0.000 | 0.000 | 0.000 | 0.000 | 0.000 |
| CBN | 0.000 | 0.000 | 0.000 | 0.000 | 0.000 | 0.000 | 0.000 | 0.000 |
| THC | 2.260 | 2.270 | 2.140 | 2.110 | 2.230 | 2.010 | 2.070 | 2.156 |
| THC-A | 0.000 | 0.000 | 0.000 | 0.000 | 0.000 | 0.000 | 0.000 | 0.000 |
| CBC | 0.000 | 0.000 | 0.000 | 0.000 | 0.000 | 0.000 | 0.000 | 0.000 |

What is claimed is:

1. A composition suitable for use in a personal vaporizer, comprising:
(a) an oil soluble liquid emulsifier base; and
(b) an extract, wherein the extract comprises a cannabinoid and a terpene.

2. The composition of claim 1, wherein the base comprises a medium chain triglyceride (MCT).

3. The composition of claim 2, wherein said MCT comprises a C-6 fatty acid, a C-8 fatty acid,
a C-10 fatty acid, a C-12 fatty acid, or a combination thereof.

4. The composition of claim 1, wherein the cannabinoid comprises tetrahydrocannabinolic acid (THCa), cannabidiolic acid (CBDa), cannabinolic acid (CBNa), cannabichromenic acid (CBCa), tetrahydrocannabinol (THC), cannabinol (CBN), cannabidiol (CBD), cannabichromene (CBC), or a combination thereof.

5. The composition of claim 4, wherein the cannabinoid comprises one or more of CBD, THC, THCa, or CBDa.

6. The composition of claim 1, wherein the composition has a combination of at least two cannabinoids.

7. The composition of claim 6, wherein the two cannabinoids are selected from a group consisting of Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM) and derivatives thereof.

8. The composition of claim 7, wherein said two cannabinoids are THC and CBD.

9. The composition of claim 6, the at least two cannabinoids are selected from tetrahydrocannabinolic acid (THCa), cannabidiolic acid (CBDa), cannabinolic acid (CBNa), cannabichromenic acid (CBCa), tetrahydrocannabinol (THC), cannabinol (CBN), cannabidiol (CBD), and cannabichromene (CBC).

10. The composition of claim 9, wherein said two cannabinoids are THCa and CBDa.

11. The composition of any one of claims 6-10, wherein the at least two cannabinoids are in a 1:1 proportion by weight.

12. The composition of any one of claims 6-10, wherein a first cannabinoid weighs about between 70 mg and 100 mg and a second cannabinoid weighs between 70 mg and 100 mg.

13. The composition of claim 12, wherein the first cannabinoid weighs about 87 mg and the second cannabinoid weighs about 87 mg.

14. The composition of any one of claims 6-10, wherein the at least two cannabinoids are in a 10:1 proportion by weight.

15. The composition of any one of claims 6-10, wherein the at least two cannabinoids are in a 20:1 proportion by weight.

16. The composition of any one of claims 6-15, wherein the total weight of cannabinoids present is between 1 and 200 mg.

17. The composition of any one of claims 6-10, wherein a first cannabinoid weighs about 180 mg and a second cannabinoid weighs about 9 mg.

18. The composition of any one of claims 6-10, wherein a first cannabinoid weighs about 171 mg and a second cannabinoid weighs about 7 mg.

19. The composition of claim 1, wherein the terpene comprises beta-myrcene, limonene, beta caryopyllene, caryopyllene oxide, terpineol, citronellol, linalool, humulene, beta-amyrin, cycloartenol, or a combination thereof.

20. The composition of claim any one of claims 1-19, wherein the extract and the base are present in an extract to base ratio of at least 9:1.

21. The composition of any one of claims 1-19, wherein the extract and the base are present in an extract to base ratio of about 7:3.

22. A method of treating a condition in a subject, comprising administering to the subject the composition of any of claims 1-21 in an amount effective to treat the condition.

23. The method of claim 22, wherein the condition is selected from the group consisting of pain associated with cancer, neuropathic pain, HIV-associated sensory neuropathy, side effects of chemotherapy, symptoms of neurology or a neurodegenerative disease, cancer, hepatitis C, methicillin-resistant Staphylococcus aureus (MRSA), pruritus, psoriasis, asthma, sickle-cell disease, sleep apnea, a digestive disease, collagen-induced arthritis, atherosclerosis and dystonia.

24. The method of claim 23, wherein the side effects of chemotherapy comprise nausea or pain.

25. The method of claim 23, wherein the symptoms of neurology or a neurodegenerative disease comprise Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, or anorexia nervosa.

26. The method of claim 23, wherein the cancer comprises a glioma, a leukemia, a skin tumor, or colorectal cancer.

27. The method of any of claims 23-26, wherein the composition is administered in a vaporized form.

28. The method of claim 27, wherein the composition is administered via a personal vaporizer.

29. A kit for administering a composition suitable for use in a personal vaporizer, the kit comprising:
a composition comprising an oil soluble liquid emulsifier base and an extract,
wherein the extract comprises a cannabinoid and a terpene;
a personal vaporizer; and instructions for the use of said kit.

30. The kit of claim 29, wherein the base comprises a medium chain triglyceride (MCT).

31. The kit of claim 30, wherein the MCT comprises a C-6 fatty acid, a C-8 fatty acid, a C-10 fatty acid, a C-12 fatty acid, or a combination thereof.

32. The kit of claim 29, wherein the cannabinoid comprises tetrahydrocannabinolic acid (THCa), cannabidiolic acid (CBDa), cannabinolic acid (CBNa), cannabichromenic acid (CBCa), tetrahydrocannabinol (THC), cannabinol (CBN), cannabidiol (CBD), cannabichromene (CBC), or a combination thereof.

33. The kit of claim 32, wherein the cannabinoid comprises one or more of CBD, THC, THCa, or CBDa.

34. The kit of claim 29, wherein the terpene comprises beta-myrcene, limonene, beta caryopyllene, caryopyllene oxide, terpineol, citronellol, linalool, humulene, beta-amyrin, cycloartenol, or a combination thereof.

35. The kit of any one of claims 29-34, wherein the extract and the base are present in an extract to base ratio of at least 9:1.

36. The kit of any one of claims 29-34, wherein the extract and the base are present in an extract to base ratio of about 7:3.

37. A cartridge for use in an electronic cigarette comprising a fluid storage compartment containing the composition of any one of claims 1-21.

38. The cartridge of claim 37, wherein the total volume of the composition present comprises 0.4 ml.

39. The cartridge of any one of claims 37-38, wherein a total number of inhalations comprises 90 inhalations per cartridge.

40. The cartridge of any one of claims 37-39, wherein each inhalation comprises 5 seconds.

41. The cartridge of any one of claims 37-39, wherein the heater of the vaporizer is maintained for 5 seconds.

42. The cartridge of any one of claims 37-41, wherein the total weight of cannabinoids delivered per 5 second inhalation comprises about 2 mg THC and about 0.1 mg CBD.

43. The cartridge of any one of claims 37-41, wherein the total weight of cannabinoids delivered per 5 second inhalation comprises 2 mg THC and 0.1 mg CBD.

44. The cartridge of any one of claims 37-41, wherein the total weight of cannabinoids delivered per 5 second inhalation comprises about 1 mg THC and about 1 mg CBD.

45. The cartridge of any one of claims 37-41, wherein the total weight of cannabinoids delivered per 5 second inhalation comprises 0.97 mg THC and 0.97 mg CBD.

46. The cartridge of any one of claims 37-41, wherein the total weight of cannabinoids delivered per 5 second inhalation comprises about .1 mg THC mg THC and about 2 mg CBD.

47. The cartridge of any one of claims 37-41, wherein the total weight of cannabinoids delivered per 5 second inhalation comprises 0.08 mg THC and 1.9 mg CBD.
